(19) United States
(12) Patent Application Publication
(10) Pub. No.: US 2005/0287106 A1
(43) Pub. Date: Dec. 29, 2005

(54) Method for shaving with at least one anhydrous film, use of at least one anhydrous film for the preparation of a shaving product, and shaving kits

(76) Inventor: Jean-Yves Legendre, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/157,081

(22) Filed: Jun. 21, 2005

Related U.S. Application Data
(60) Provisional application No. 60/587,058, filed on Jul. 13, 2004.

(30) Foreign Application Priority Data
Jun. 22, 2004 (FR) 04 51191

(51) Int. Cl. A61K 7/15

(52) U.S. Cl. 424/73

(57) ABSTRACT

Disclosed herein is a method for shaving the skin, comprising (1) forming at the time of use a shaving composition by bringing at least one anhydrous film into contact with a solvent, for example an aqueous composition, (2) applying the composition thus formed to the surface of the skin to be shaved, and (3) shaving hairs using a razor. Also disclosed herein is a shaving kit comprising (a) at least one anhydrous film which is soluble or dispersible in a solvent, (b) at least one razor, such as a disposable razor, and/or (c) a means for spreading a shaving composition. The kit may further comprise the solvent intended to be mixed at the time of use with the anhydrous film or an aftershave composition.

This application claims benefit of U.S. Provisional Application No. 60/587,058, filed Jul. 13, 2004, the contents of which are incorporated herein by reference. This application also claims benefit of priority under 35 U.S.C. § 119 to French Patent Application No. 04 51191, filed Jun. 22, 2004, the contents of which are also incorporated by reference.

Disclosed herein is a shaving method comprising generating a shaving composition by bringing at least one anhydrous film which is soluble or dispersible in a solvent into contact with the solvent.

Also disclosed herein are shaving kits comprising at least one anhydrous film which is soluble or dispersible in a solvent and at least one razor, for example a disposable razor, and/or a means for spreading a shaving composition.

The products used by a man to remove hairs from his body for aesthetic or hygienic purposes are known and varied, from shaving soap, whether powder, bar, or cake, oils up to shaving creams and foams and up to aqueous shaving gels (refer in this respect to the article entitled “Shaving Preparations—Origin of Shaving and Sales Trends”, by Robert E. Sauté, published in the work “The Chemistry and Manufacture of Cosmetics”, Second Edition, 1975, Vol. IV, chapter 64, pages 1313 to 1341, from Maison G. de Navarre).

In addition to conventional shaving gels, transparent non-foaming gels and shaving gels with delayed foaming (or with a delayed foaming effect) are also known and have been disclosed in numerous patents, such as, for example, U.S. Pat. Nos. 3,541,581 and 4,405,489 and French Patent No. FR 2 595 943. These gels with a delayed foaming effect are compositions that may be packaged under pressure in an aerosol device which delivers; under the effect of a propellant, gels which are non-foaming under static conditions but which, under the mechanical action due to the spreading of the product on the skin, may spontaneously and virtually instantaneously generate a foam on the skin.

Current shaving products may be packaged in multidose form, such as jars, bottles, tubes and aerosols. These packaging forms may not be suitable for travel as they are bulky and, for some of them, easily damaged. Small aerosols have been developed to meet this need but they may be expensive to manufacture. Articles of shaving paper or wipe type have been provided and are, for example, disclosed in the Japanese patent documents JP 2000-319147, JP 09-157143, and JP 2000-256164 and U.S. patent application Publication Ser. No. 2001/0046513. These articles may be supports impregnated with a fluid formulation which may facilitate shaving. Apart from the fact that these products may result in problems of biodegradability which may be harmful to the environment, they may be very difficult to use on unshaven skin.

This is because shaving products should ideally exhibit an appropriate texture which may make possible good spreading over the skin of the face, which may facilitate the movement of the razor over the surface of the skin to be treated, and which may facilitate the cutting of the individual hair. In addition, a shaving formulation should ideally be able to rapidly develop the desired texture in a homogeneous and stable way on contact with water. Finally, the shaving product should ideally have good cosmetic qualities, such as at least one of moisturizing, contribution of softness, comfort on shaving, absence of tacky effect, and absence of runny effect on the skin. The shaving product should also be easily removed on rinsing with water, both from the skin and from the razor blades.

The need thus exists to have available novel forms of shaving products which may be suitable for travel, which are small in volume, which are inexpensive to manufacture, which are easy to employ, and/or which may exhibit at least one of the qualities of a good shaving product as mentioned above.

Thus, after much research, the present inventor has now discovered that it is possible to obtain a shaving formulation which meets this need starting from at least one anhydrous film which is dispersible or soluble in a solvent such as an aqueous composition. Such an at least one anhydrous film is intended to form at the time of use, on contact with the solvent, a composition capable of being applied to the skin and of making possible effective and comfortable shaving.

This discovery is at the basis of at least one embodiment of the present disclosure.

Admittedly, the use is known of anhydrous films for immediate dissolution on contact with water or saliva and of edible films targeted at wrapping foods in order to increase their storage life, and this technique has formed the subject-matter of numerous applications (Guilbert S. et al., Technology and applications of edible protective films, Packaging Technology and Science, vol. 8, pp. 339-346, 1995) and of patents, such as, for example, U.S. Pat. Nos. 5,965,708 and 5,962,053 and Japanese Patent No. JP A 10/215792. Likewise, this type of technology is used in the pharmaceutical field for administering active principles by the oral route in the form of formulations for instantaneous buccal disintegration, for example as disclosed in the patent documents WO A 2002/085119, WO A 2002/043657, and WO A 2001/070194. The use of these films has also been extended to the administration of medicaments to other mucous membranes, such as the vagina (see, for example, EP A 1 110 546) and to wounds (see, for example, JP A 63/220876).

In addition, the Japanese patent document JP A 2002/212027 discloses the production and the composition of cosmetic preparations in the form of water-soluble films without claiming any specific use. International patent documents WO A 2002/05789 and WO-A-2003/075812 and U.S. patent application Publication Ser. No. 2002/0127254 disclose the preparation and administration of anhydrous polymeric films for direct administration of cosmetic compositions to the prewetted skin. Furthermore, U.S. patent application Publication Ser. No. 2003/0186826 discloses a dry cosmetic composition based on polymers and on surfactants to be administered to the skin or hair with water. However, these documents never envisage the preparation at the time of use of a product intended for shaving the skin.

The use in cosmetics of hydratable films based on modified starch and on an active principle is disclosed, for example, in European Patent Application No. EP 1 317 916, for example in hair compositions such as shampoos, hair fixing compositions, deodorant products, and washing products. However, this document never envisages the preparation at the time of use of a product intended for shaving the skin.

In addition, the use is known, for example as disclosed in U.S. patent application Publication. Ser. No. 2004/0029762, of hydratable sheets of soap or hydratable sheets comprising a high concentration of surfactants intended for cleaning the skin and skin hygiene. Water-soluble sheets based on water-soluble polymer, on poly(vinyl alcohol) and on a surfactant which are intended for cleaning the skin and skin hygiene are also disclosed, for example, in patent application Ser. No. WO 2004/032859. These sheets are not described for use in shaving the skin.

Thus, disclosed herein is a method for shaving the skin, comprising:

(1 ) generating at the time of use a shaving composition by bringing at least one anhydrous film which is soluble or dispersible in a solvent into contact with the solvent, then

(2) applying the shaving composition thus formed to the surface of the skin to be shaved, and

(3) shaving the skin using a razor.

Another embodiment disclosed herein relates to the use of at least one anhydrous film which is soluble or dispersible in a solvent, such as an aqueous solution, to prepare a composition for shaving the skin.

Further disclosed herein is a shaving kit comprising

(a) at least one anhydrous film which is soluble or dispersible in a solvent such as an aqueous composition,

(b) at least one razor, such as a disposable razor, and/or

(c) a means for spreading a shaving composition, for example, a brush such as a shaving brush, or a sponge.

The solvent used in accordance with certain embodiments may be an organic solvent, such as, for example, an oil or a mixture of oils commonly used in the manufacture of shaving oils, or else a composition comprising at least one organic solvent and optionally comprising at least one cosmetic active principle for the comfort and/or care of the skin.

The solvent used in accordance with certain embodiments disclosed herein may be an aqueous composition and the at least one anhydrous film will be soluble or dispersible in water. The aqueous composition may be chosen from tap water, thermal water, spring water, scented water, and aqueous compositions comprising at least one cosmetic active principle for the comfort and/or care of the skin.

As used herein, the term “film” is understood to mean a thin solid which can be grasped. As used herein, the term “thin” is understood to mean a solid having a thickness of at most 5,000 μm.

The at least one anhydrous film can be formed of a single layer or can be composed of several superimposed layers.

The at least one anhydrous film in accordance with the present disclosure should have an appropriate size in order to be able to be easily handled by the user. The film may be in the shape of a square, rectangle, disc, or any other shape. Each film may have a thickness ranging from 50 μm to 5,000 μm, such as 100 to 2,000 μm. Each film may have a surface area ranging from 1 to 50 cm², such as from 2 to 30 cm².

Furthermore, as used herein the term “anhydrous film” is understood to mean a film comprising less than 20% by weight of water, such as less than 10% by weight, with respect to the total weight of the film.

As used herein, the term “soluble film” is understood to mean a film which dissolves in the solvent.

As used herein, the term “dispersible film” is understood to mean a film which disperses in the solvent.

According to one embodiment, the at least one anhydrous film disclosed herein comprises at least one hydrophilic polymer which is soluble in water and fibers which are soluble or dispersible in water.

Mention may be made, as examples of water-soluble hydrophilic polymers which can be used according to certain embodiments, of the following polymers:

polyvinylpyrrolidones;

cellulose polymers, such as hydroxyethyl cellulose, hydroxypropyl cellulose, hydroxypropyl methyl cellulose, methylcellulose, ethylhydroxyethyl cellulose, carboxymethyl cellulose, and quaternized derivatives of cellulose; and

optionally modified starches, such as those disclosed in European Patent Application No. EP 1 317 916;

optionally modified polymers of natural origin, such as gum arabic, guar gum, xanthan derivatives, karaya gum, glycoaminoglycans, shellac, sandarac gum, dammers, elemis, copals, pectin, mannan, galactomannans, glucomannans, and derivatives thereof, and

mixtures of these polymers.

The at least one hydrophilic polymer may be present in the film in an amount ranging from 5 to 80% by weight, such as from 10 to 60% by weight, with respect to the total weight of the film.

The fibers which can be used in the composition disclosed herein may be chosen from fibers of natural origin, such as cellulose fibers extracted from plants and, for example, cellulose fibers resulting from at least one of wood, silk, cotton, wool, flax, fruit, and beet.

In at least one embodiment, the fibers of natural origin may be chosen from wood cellulose fibers in the form of pulp. Mention may be made, among these fibers, of the products corresponding, for example, to the commercial references Lignocel®, Lyocell, Rayon Flock, Natural Rayon, Cell-U-Lash, AV Cell, Biofluff, Temfilm, and Biofloc®.

Polylactic acid (PLA) fibers resulting from maize may also be used.

The fibers may be introduced into the at least one anhydrous film in the form of paper which may be dissolved or dispersed in water. Such products are known, for example, under the trade names of Dissolvo® (Gilbreth) and MDP (Mishima Paper).

The fibers of natural origin disclosed herein can be mixed with synthetic resins or fibers, such as polypropylene, polyethylene, poly(ethylene terephthalate), polyamide, or poly(vinyl acetate) resins or fibers.

Mention may be made, among water-soluble fibers, of the fibers produced from poly(vinyl alcohol) (PVA), such as, for example, the products sold by the Japanese company Kuraray under the name Kuralon K II® WN2. The process for the manufacture of these fibers may comprise using organic solvents. The cross section of these fibers may be substantially circular. European Patent Application No. EP 636 716 discloses water-soluble PVA-based fibers and their process of preparation.

Mention may also be made, among water-soluble fibers, of poly(ethylene oxide) fibers; polylactic acid fibers, such as the Lactron® fibers from the Japanese company Kanebo; and the polysaccharide fibers sold under the name Lysorb® by Lysac Technologies Inc.

The fibers may be present in the film in an amount ranging from 1 to 90% by weight, such as from 5 to 80% by weight, relative to the total weight of the film.

According to one embodiment disclosed herein, the at least one anhydrous film further comprises at least one surfactant.

The at least one surfactant may be chosen from anionic, amphoteric, zwitterionic, or nonionic surfactants and mixtures thereof.

Mention may be made, among the anionic surfactants which can be used according to certain embodiments, of salts of fatty acids (or soaps) comprising a saturated or unsaturated, linear or branched, alkyl chain having from 6 to 30 carbon atoms, for example having 12 to 22 carbon atoms.

The bases that may be used to completely neutralize these fatty acids may be chosen from inorganic bases, such as alkali metal hydroxides (sodium hydroxide and potassium hydroxide), alkaline earth metal (magnesium) hydroxides, and aqueous ammonia; organic bases, such as alkanolamines, for example triethanolamine, monoethanolamine, and monoisopropanolamine; and amino acids, such as N-methylglucamine, lysine, and arginine.

As examples of fatty acid salts, mention may be made of alkanolamine (i.e., triethanolamine) salts or potassium hydroxide salts of lauric acid, myristic acid, palmitic acid, and stearic acid, and mixtures thereof.

Mention may be made, as other anionic surfactants which can be used in accordance with certain embodiments, of salts, for example, alkali metal salts, such as sodium salts, ammonium salts, amine salts, aminoalcohol salts, and alkaline earth metal salts, for example magnesium salts, of the following types: alkyl sulphates, alkyl ether sulphates, alkylamido ether sulphates, alkylaryl polyether sulphates, monoglyceride sulphates, alkylsulphonates, alkylamidesulphonates, alkylarylsulphonates, α-olefinsulphonates, paraffinsulphonates, alkyl sulphosuccinates, alkyl ether sulphosuccinates, alkylamide sulphosuccinates, alkyl sulphoacetates, acylsarcosinates, and acylglutamates, the alkyl and acyl groups of all these compounds comprising from 6 to 24 carbon atoms and the aryl group denoting, for example, a phenyl or benzyl group. Use may also be made of C₆-C₂₄ alkyl esters of polyglycosidecarboxylic acids, such as alkyl glucosidecitrates, alkyl polyglycosidetartrates, and alkyl polyglycosidesulphosuccinates; alkyl sulphosuccinamates, acyl isethionates, and N-acyltaurates, the alkyl or acyl group of all these compounds comprising from 12 to 20 carbon atoms. Mention may also be made, among the anionic surfactants which may be used, of the acyl lactylates, the acyl group of which comprises from 8 to 20 carbon atoms, and mixtures thereof.

In addition, mention may also be made of alkyl-D-galactosideuronic acids, polyoxyalkylenated (C₆-C₂₄)alkyl ether carboxylic acids, polyoxyalkylenated (C₆-C₂₄) alkyl(C₆-C₂₄)aryl ether carboxylic acids, polyoxyalkylenated (C₆₋₂₄)alkylamido ether carboxylic acids, such as those comprising from 2 to 50 ethylene oxide groups, and their alkali metal, ammonium, amine, aminoalcohol, and alkaline earth metal salts.

The at least one amphoteric surfactant which may be suitable according to the present disclosure may be chosen from, for example, derivatives of aliphatic secondary or tertiary amines in which the aliphatic group is a linear or branched chain comprising from 8 to 22 carbon atoms and comprising at least one water-solubilizing anionic group, such as, for example, carboxylate, sulphonate, sulphate, phosphate, and phosphonate groups.

Mention may be made, among the amine derivatives, of the products sold under the name Miranol®, as disclosed in U.S. Pat. Nos. 2,528,378 and 2,781,354 and classified in the CTFA dictionary, 3rd edition, 1982, under the names Amphocarboxyglycinate and Amphocarboxypropionate.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lau roamphodipropion ic acid, and Cocoamphodipropionic acid. Mention may be made, by way of example, of the cocoamphodiacetate sold by Rhodia under the trade name Miranol® C2M concentrate.

The at least one amphoteric surfactant which may be suitable in accordance with the present disclosure may also be chosen from (C₈-C₂₀)alkylbetaines, sulphobetaines, (C₈-C₂₀)alkylamido(C₆-C₈)alkylbetaines, (C₈-C₂₀)alkylamido(C₆-C₈)alkylsulphobetaines, and mixtures thereof, for example (C₈-C₂₀)alkylbetaines, such as cocobetaine, sold under the trade names Mirataine® BB/FLA of Rhodia and Empigen® BB/FL of Huntsman.

The at least one nonionic surfactant may be chosen, for instance, from polyethoxylated, polypropoxylated, and polyglycerolated fatty acids; (C₁-C₂₀)alkylphenols; α-diols; and alcohols having a fatty chain comprising, for example, from 8 to 18 carbon atoms, it being possible for the number of ethylene oxide or propylene oxide groups to range, for example, from 2 to 50 and it being possible for the number of glycerol groups to range, for example, from 2 to 30. Mention may also be made of copolymers of ethylene oxide and of propylene oxide, condensates of ethylene oxide and of propylene oxide with fatty alcohols; polyethoxylated fatty amides, for example having from 2 to 30 mol of ethylene oxide, polyglycerolated fatty amides comprising on average from 1 to 5 glycerol groups, such as from 1.5 to 4 glycerol groups; polyethoxylated fatty amines for example having 2 to 30 mol of ethylene oxide; sorbitan ethoxylated fatty acid esters having from 2 to 30 mol of ethylene oxide; sucrose fatty acid esters; polyethylene glycol esters of fatty acids; (C₆-C₂₄)alkylpolyglycosides; N-(C₆-C₂₄)alkylglucamine derivatives; amine oxides, such as oxides of (C₁₀-C₁₄)alkylamines and N-(C₁₀-C₁₄)acylaminopropylmorpholine oxides; and mixtures thereof.

The at least one surfactant may be present in the at least one anhydrous film in an amount ranging from 5 to 80% by weight, such as from 10 to 60% by weight, relative to the total weight of the film.

In at least one embodiment, the at least one surfactant may be chosen from anionic surfactants, for example salts of fatty acids (soaps), such as those defined above.

The at least one anhydrous film disclosed herein may additionally comprise at least one plasticizing agent.

The at least one plasticizing agent may be chosen from polyols, such as glycerol, polyethylene glycol, dipropylene glycol, butylene glycol, and pentylene glycol; sugar alcohols, such as sorbitol, mannitol, maltitol, and lactitol; mono-, di-, and oligosaccharides, such as glucose, fructose, sucrose, maltose, and lactose; polycarboxylic acids, such as citric acid, maleic acid, succinic acid, polyacrylic acid, and polymaleic acid; and polyesters, such as glyceryl triacetate, acetylated monoglyceride, diethyl phthalate, triethyl citrate, tributyl citrate, triethyl acetylcitrate, and tributyl acetylcitrate.

The at least one anhydrous film disclosed herein may further comprise at least one other ingredient or active principle known in the field of shaving products.

Mention may be made, for example, of:

moisturizing agents, and among them glycerol and sorbitol;

soothing agents, such as allantoin or α-bisabolol;

refreshing and scenting agents, such as menthol and its derivatives;

lubricants, such as silicones, polydecenes, polyvinylpyrrolidones, and cationic polymers;

superfatting agents, such as waxes, oils of vegetable, animal, or mineral origin, esters of fatty acids, fatty alcohols, and fatty acids;

emollients, such as polyethylene glycol, polypropylene glycol, the benzoate of C₁₂/C₁₅ alcohols, glycerol stearate, and polyethylene glycol stearate;

opacifying agents;

stabilizing agents;

colorants;

fragrances;

preservatives;

antioxidants;

pH-regulating agents; and

active compounds.

Mention may be made, among active compounds, of vitamins, such as vitamin E (tocopherol) and its derivatives; vitamin A (retinol) and its derivatives; polyunsaturated fatty acids; essential oils; α-hydroxy acids, such as lactic acid and glycolic acid; ascorbic acid (vitamin C) and its derivatives; urea; amino acids; oligopeptides; plant extracts, such as those of ginkgo biloba; peptide hydrolysates, protein hydrolysates; trace elements; hyaluronic acid and its salts; panthenol; β-hydroxy acids, such as acetylsalicylic acid; and mixtures thereof.

Of course, a person skilled in the art will take care to choose the at least one optional additional compound mentioned above so that the advantageous properties intrinsically attached to the at least one anhydrous film disclosed herein is not, or not substantially, detrimentally affected by the envisaged addition or additions.

The at least one anhydrous film disclosed herein may be devoid of preservative. This may be advantageous in terms of harmlessness.

In certain embodiments, the at least one anhydrous film disclosed herein may be used singly and the at least one anhydrous film may exhibit excellent preservation, even without addition of preservative, which may be by virtue of its dry nature.

The at least one anhydrous film disclosed herein may be packaged in an article which may facilitate the grasping thereof, such as that disclosed in French Patent Application No. FR 0 351 002, the content of which is incorporated herein by reference. The at least one anhydrous film may be packaged in, for example, a plastic dispensing box, an individual bag, or a blister pack. The at least one anhydrous film disclosed herein, which is hydratable, may be packaged, for example, in a case with a drawer or with a lid articulated on a base. The case can comprise means intended to facilitate the dispensing of the articles. The dispensing means can be of the type disclosed, for example, in U.S. Pat. No. 2,973,882, British Patent No. GB A 2 358 627, Swiss Patent No. CH A 461 025, and U.S. Pat. No. 6,578,732.

Further disclosed herein is a shaving kit comprising

(a) at least one anhydrous film which is soluble or dispersible in a solvent, such as an aqueous composition, and

(b) at least one razor, such as a disposable razor, and/or

(c) a means for spreading a shaving composition, such as, for example, a brush, for example a shaving brush, or a sponge.

According to one embodiment, the shaving kit may further comprise the solvent intended to be mixed at the time of use with the at least one anhydrous film of the same kit, for example the aqueous composition.

The solvent, such as the aqueous composition, can be provided in a single-dose form, such as bags, tubes, vials, prefilled syringes, soft capsules, shells, or trays made of thermoformed plastic. The aqueous composition may also be provided in a conventional multi-dose form, such as a bottle, and may be packaged in a system dispensing a predefined dose. Such a system may, for example, be chosen from pump-action spray systems, aerosol systems, pipette systems, graduated syringe systems, and dropper systems.

According to another embodiment disclosed herein, the shaving kit may further comprise, in addition, at least one aftershave composition.

Here again, the at least one aftershave composition can be provided in a single-dose form, such as bags, impregnated wipes, tubes, vials, prefilled syringes, soft capsules, shells, or trays made of thermoformed plastic. The at least one aftershave composition may also be provided in a conventional multi-dose form, such as a bottle, and may be packaged in a system dispensing a predefined dose. Such a system may be chosen from pump-action spray systems, aerosol systems, pipette systems, graduated syringe systems, and dropper systems.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

Concrete but in no way limiting examples illustrating certain embodiments will now be given.

### EXAMPLE 1

A water-soluble anhydrous film with the following composition was prepared:

| | |
|---|---|
| Modified starch | 30% by weight |
| Wood cellulose pulp | 25% by weight |
| Soap | 35% by weight |
| Glycerol | 10% by weight |

After bringing the film into contact with tap water, a foaming gel was instantaneously obtained, the texture of which made possible ready spreading over the skin of the face and effective and comfortable shaving.

1. A method for shaving the skin, comprising:
(1) generating a shaving composition by bringing at least one anhydrous film which is soluble or dispersible in a solvent into contact with the solvent,
(2) applying the shaving composition thus formed to the surface of skin to be shaved, and
(3) shaving the skin using a razor.

2. The method according to claim 1, wherein the at least one anhydrous film has a thickness ranging from 50 μm to 5,000 μm.

3. The method according to claim 2, wherein the at least one anhydrous film has a thickness ranging from 100 to 2,000 μm.

4. The method according to claim 1, wherein the at least one anhydrous film is formed by a single layer.

5. The method according to claim 1, wherein the at least one anhydrous film is composed of more than one superimposed layer.

6. The method according to claim 1, wherein the at least one anhydrous film has a surface area ranging from 1 to 50 cm².

7. The method according to claim 6, wherein the at least one anhydrous film has a surface area ranging from 2 to 30 cm².

8. The method according to claim 1, wherein the at least one anhydrous film is dispersible or soluble in water and the solvent is an aqueous composition.

9. The method according to claim 1, wherein the at least one anhydrous film comprises at least one hydrophilic polymer which is soluble in water and fibers which are soluble or dispersible in water.

10. The method according to claim 9, wherein the at least one hydrophilic polymer which is soluble in water is chosen from:
polyvinylpyrrolidones;
cellulose polymers;
optionally modified starches; and
optionally modified polymers of natural origin.

11. The method according to claim 9, wherein the at least one hydrophilic polymer which is soluble in water is present in the at least one anhydrous film in an amount ranging from 5 to 80% by weight, relative to the total weight of the film.

12. The method according to claim 11, wherein the at least one hydrophilic polymer which is soluble in water is present in the at least one anhydrous film in an amount ranging from 10 to 60% by weight, relative to the total weight of the film.

13. The method according to claim 9, wherein the fibers comprise fibers of natural origin.

14. The method according to claim 13, wherein the fibers of natural origin are chosen from at least one of silk, cotton, wool, flax, cellulose fibers extracted from plants, and cellulose fibers resulting from wood.

15. The method according to claim 13, wherein the fibers of natural origin are mixed with synthetic resins or fibers.

16. The method according to claim 15, wherein the synthetic resins or fibers are chosen from at least one of polypropylene, polyethylene, poly(ethylene terephthalate), polyamide, and poly(vinyl acetate) resins or fibers.

17. The method according to claim 13, wherein the fibers are water-soluble fibers produced from poly(vinyl alcohol) (PVA).

18. The method according to claim 13, wherein the fibers are present in the at least one anhydrous film in an amount ranging from 1 to 90% by weight, relative to the total weight of the film.

19. The method according to claim 18, wherein the fibers are present in the at least one anhydrous film in an amount ranging from 5 to 80% by weight, relative to the total weight of the film.

20. The method according to claim 1, wherein the at least one anhydrous film further comprises at least one surfactant.

21. The method according to claim 20, wherein the at least one surfactant is chosen from anionic, amphoteric, zwitterionic, and nonionic surfactants and mixtures thereof.

22. The method according to claim 21, wherein the at least one surfactant is chosen from anionic surfactants.

23. The method according to claim 21, wherein the at least one surfactant is chosen from salts of fatty acids comprising at least one saturated or unsaturated, linear or branched, alkyl chain having from 6 to 30 carbon atoms.

24. The method according to claim 23, wherein the at least one saturated or unsaturated, linear or branched alkyl chain has 12 to 22 carbon atoms.

25. The method according to claim 23, wherein the salts of fatty acids are chosen from alkanolamine salts; potassium hydroxide salts of lauric acid, myristic acid, palmitic acid, and stearic acid; and mixtures thereof.

26. The method according to claim 20, wherein the at least one surfactant is present in the film in an amount ranging from 5 to 80% by weight, with respect to the total weight of the film.

27. The method according to claim 26, wherein the at least one surfactant is present in the film in an amount ranging from 10 to 60% by weight, with respect to the total weight of the film.

28. The method according to claim 1, wherein the at least one anhydrous film further comprises at least one plasticizing agent.

29. The method according to claim 1, wherein the at least one anhydrous film further comprises at least one additive chosen from moisturizing agents; soothing agents; refreshing agents; scenting agents; lubricants; superfatting agents; emollients; opacifying agents; stabilizing agents; colorants; fragrances; preservatives; antioxidants; pH-regulating agents; and active compounds.

30. The method according to claim 29, wherein the active compounds are chosen from vitamin E (tocopherol) and its derivatives; vitamin A (retinol) and its derivatives; polyunsaturated fatty acids; essential oils; α-hydroxy acids; ascorbic acid (vitamin C) and its derivatives; urea; amino acids; oligopeptides; plant extracts; peptide hydrolysates; protein hydrolysates; trace elements; hyaluronic acid and its salts; panthenol; β-hydroxy acids; and mixtures thereof.

31. The method according to claim 30, wherein the α-hydroxy acids are chosen from lactic acid and glycolic acid.

32. The method according to claim 30, wherein the plant extracts are chosen from ginkgo biloba.

33. The method according to claim 30, wherein the β-hydroxy acids are chosen from acetylsalicylic acid.

34. A shaving kit, comprising:
(a) at least one anhydrous film which is soluble or dispersible in a solvent and which forms a shaving composition when brought into contact with the solvent, and
(b) at least one razor, and/or
(c) a means for spreading the shaving composition.

35. The shaving kit according to claim 34, wherein the at least one razor is a disposable razor.

36. The shaving kit according to claim 34, further comprising the solvent intended to be mixed at the time of use with the at least one anhydrous film.

37. The shaving kit according to claim 34, further comprising at least one aftershave composition.
